(19) United States
(12) Patent Application Publication
(10) Pub. No.: US 2006/0047003 A1
(43) Pub. Date: Mar. 2, 2006

(54) Fullerene compositions for ameliorating hearing loss, collateral damage of chemotherapy, or mucositis

(76) Inventor: Russ Lebovitz, Houston, TX (US)

Correspondence Address: WILLIAMS, MORGAN & AMERSON, 10333 RICHMOND, SUITE 1100, HOUSTON, TX, 77042, US

(21) Appl. No.: 11/214,469

(22) Filed: Aug. 29, 2005

Related U.S. Application Data
(60) Provisional application No. 60/606,779, filed on Sep. 2, 2004.

(51) Int. Cl. A61K 31/192; A61K 31/165

(52) U.S. Cl. 514/569; 514/617

(57) ABSTRACT

This patent discloses a method of ameliorating a hearing loss, collateral damage of chemotherapy, or mucositis in a mammal, comprising administering a composition comprising a substituted fullerene to the mammal afflicted with the hearing loss, collateral damage of chemotherapy, or mucositis or potentially afflicted with the hearing loss, collateral damage of chemotherapy, or mucositis wherein the substituted fullerene comprises a fullerene core (Cn) and at least one of: (i) from 1 to 3 (>CX¹X²) groups bonded to the fullerene core; (ii) from 1 to 18 —X³ groups bonded to the fullerene core; (iii) from 1 to 6 —X⁴-groups bonded to the fullerene core; or (iv) from 1 to 6 dendrons bonded to the fullerene core.

This application claims priority from prior copending U.S. provisional patent application Ser. No. 60/606,779, filed on Sep. 2, 2004.

### BACKGROUND OF THE INVENTION

The present invention relates generally to the fields of ameliorating hearing loss, collateral damage of chemotherapy, or mucositiss. More particularly, it concerns the use of fullerene compositions to treat hearing loss, collateral damage of chemotherapy, or mucositis.

Reactive oxygen species (ROS), commonly referred to as “free radicals,” have been implicated in a variety of diseases. ROS are believed to promote, in at least certain cells, cell types, tissues, or tissue types, cell death (apoptosis), impaired cellular function, and modification or change in proportion of extracellular matrix components such as elastin or collagen, among other symptoms.

A need exists to treat diseases by administering compounds that can neutralize ROS.

### SUMMARY OF THE INVENTION

In one embodiment, the present invention relates to administering a composition comprising a substituted fullerene to a mammal afflicted with hearing loss, collateral damage of chemotherapy, or mucositis or potentially afflicted with hearing loss, collateral damage of chemotherapy, or mucositis, wherein the substituted fullerene comprises a fullerene core (Cn) and at least one of:

(i) from 1 to 3 (>CX¹X²) groups bonded to the fillerene core;

(ii) from 1 to 18 —X³ groups bonded to the fullerene core;

(iii) from 1 to 6 —X⁴ groups bonded to the fullerene core; or

(iv) from 1 to 6 dendrons bonded to the fullerene core.

Herein, the word “or” has the inclusive meaning wherever it appears.

In another embodiment, the present invention relates to a composition for ameliorating a hearing loss, collateral damage of chemotherapy, or mucositis in a mammal, comprising (a) a substituted fullerene; and (b) a pharmaceutically acceptable carrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A shows an exemplary substituted fullerene in structural formula, and FIG. 1B shows the same substituted fillerene in a schematic formula.

FIG. 2 shows the decarboxylation of C3 to C3-penta-acid and thence to C3-tetra-acid.

FIG. 3 shows the decarboxylation of C3-tetra-acid to C3-tris-acid.

FIG. 4 shows the chirality of C3.

FIG. 5 shows the effect of C3 chirality on isomers formed by decarboxylation.

FIG. 6 shows exemplary substituted fullerenes according to one embodiment of the present invention. C3, in the upper left, is comparative.

FIGS. 7A and 7B show two exemplary substituted fullerenes.

FIGS. 8A-8G show seven exemplary dendrofullerenes.

FIG. 9 shows dendrofullerene DF-1.

FIGS. 10A-10H report the IC50 values for various substituted fullerenes (and Trolox, a known non-fullerene antioxidant), as described in Example 1.

### DESCRIPTION OF ILLUSTRATIVE EMBODIMENTS

In one embodiment, the present invention relates to a method of ameliorating hearing loss, collateral damage of chemotherapy, or mucositis in a mammal, comprising:

administering a composition comprising a substituted fullerene to the mammal afflicted with the hearing loss, collateral damage of chemotherapy, or mucositis or potentially afflicted with the hearing loss, collateral damage of chemotherapy, or mucositis.

By “ameliorating” a disease is meant improving the condition of a subject suffering or at risk of suffering from the disease. Ameliorating can comprise one or more of the following: a reduction in the severity of a symptom of the disease, a reduction in the extent of a symptom of the disease, a reduction in the number of symptoms of the disease, a reduction in the number of disease agents, a reduction in the spread of a symptom of the disease, a delay in the onset of a symptom of the disease, a delay in disease onset, or a reduction in the time between onset of the disease and remission of the disease, among others apparent to the skilled artisan having the benefit of the present disclosure. To the extent that the foregoing examples of ameliorating a disease are defined in relative terms, the proper comparison is to the disease or symptoms thereof when no composition is administered to ameliorate it and no method is performed to ameliorate it. The terms “preventing” (herein meaning “to stop a disease from onsetting”) and “treating” (herein meaning “to improve the condition of a mammal suffering from a disease”) are both within the scope of “ameliorating,” as used herein.

In the present invention, the disease is an oxidative stress disease. An “oxidative stress disease” is a disease in which the healthy function of one or more organelles, non-organelle subcellular structures, cells, cell types, tissues, tissue types, organs, or organ systems is impaired by the action of oxidizing agents, such as free radicals, particularly radical oxygen species (ROS). The action of oxidizing agents need not be the only route by which impairment of healthy function occurs in the course of a disease for the disease to be an oxidative stress disease. In oxidative stress diseases, a number of sources of oxidizing agents are known. Exemplary sources include, but are not limited to, by-processes of metabolism, irritation by environmental agents (for example, tobacco smoke or ultraviolet light), or internal challenge (for example, ischemia), among others. Any one or more of a large number of oxidative stress diseases can be ameliorated by performance of the method.

Hearing loss refers to a state wherein the minimum audible threshold (in dB) of a sound of a particular frequency to a mammal is increased relative to an initial state.

Collateral damage of chemotherapy refers to injuries suffered by healthy tissues of a mammal upon exposure to cytotoxic drugs. Generally, chemotherapy is used in treating certain cancers, but this is not a limitation of the present invention.

Mucositis refers to a fungal infection of a mucous membrane. Fungal infections of mucous membranes are most common among immunocompromised individuals, such as people suffering from HIV infection or certain cancers or undergoing immunosuppressant therapy to combat rejection of transplanted organs, among others. However, fungal infections of the mucous membranes of any mammal are within the scope of “mucositis,” as the term is used herein.

In any of the foregoing, the oxidative stress disease inflicts one or more of cell death, cell injury, impaired cell function, the production of cellular products reflective of cell injury, the proliferation of cell types not normally present in a tissue or not normally present in a tissue at such high levels, the degradation or alteration of extracellular matrix, or other symptoms generally recognizable by the skilled artisan as indicating an oxidative stress disease, on the mammal.

Any mammal which suffers a hearing loss, collateral damage of chemotherapy, or mucositis, as defined herein, can receive the administered composition. An exemplary mammal is Homo sapiens, although other mammals possessing economic or esthetic utility (e.g., livestock such as cattle, sheep, or horses; e.g., pets such as dogs and cats) can receive the administered composition.

The term “administering,” as used herein, is intended to encompass all techniques of introducing a composition to a mammal. Exemplary routes of administration include intravenous, intraarterial, intramuscular, subcutaneous, oral, rectal, nasal, or topical, among others.

The composition comprises a substituted fullerene.

Buckminsterfullerenes, also known as fullerenes or, more colloquially, buckyballs, are cage-like molecules consisting essentially of sp²-hybridized carbons and have the general formula (C₂₀₊₂ₘ) (where m is a natural number). Fullerenes are the third form of pure carbon, in addition to diamond and graphite. Typically, fullerenes are arranged in hexagons, pentagons, or both. Most known fullerenes have 12 pentagons and varying numbers of hexagons depending on the size of the molecule. “Cₙ” refers to a fullerene moiety comprising n carbon atoms.

Common fullerenes include C₆₀ and C₇₀, although fullerenes comprising up to about 400 carbon atoms are also known.

Fullerenes can be produced by any known technique, including, but not limited to, high temperature vaporization of graphite. Fullerenes are available from MER Corporation (Tucson, Ariz.) and Frontier Carbon Corporation, among other sources.

A substituted fullerene is a fullerene having at least one substituent group bonded to at least one carbon of the fullerene core. Exemplary substituted fullerenes include carboxyfullerenes and hydroxylated fullerenes, among others.

A carboxyfullerene, as used herein, is a fullerene derivative comprising a Cₙ core and one or more substituent groups, wherein at least one substituent group comprises a carboxylic acid moiety or an ester moiety. Generally, carboxyfullerenes are water soluble, although whether a specific carboxyfullerene is water soluble is a matter of routine experimentation for the skilled artisan.

In another embodiment, the fullerene is a hydroxylated fullerene. A “hydroxylated fullerene,” as used herein, is a fullerene derivative comprising a Cₙ core and one or more substituent groups, wherein at least one substituent group comprises a hydroxyl moiety.

In all embodiments, the substituted fullerene comprises a fullerene core (Cn), which can have any number of carbon atoms. In one embodiment, the Cn has 60 carbon atoms (and may be represented herein as C₆₀). In one embodiment, the Cn has 70 carbon atoms (and may be represented herein as C₇₀).

Throughout this description, particular embodiments described herein may be described in terms of a particular acid, amide, ester, or salt conformation, but the skilled artisan will understand an embodiment can change among these and other conformations depending on the pH and other conditions of manufacture, storage, and use. All such conformations are within the scope of the appended claims. The conformational change between, e.g., an acid and a salt is a routine change, whereas a structural change, such as the decarboxylation of an acid moiety to —H, is not a routine change.

In one embodiment, the substituted fullerene comprises a fullerene core (Cn) and from 1 to 3 (>CX¹X²) groups bonded to the fullerene core. The notation “>C” indicates the group is bonded to the fullerene core by two single bonds between the carbon atom “C” and the Cn. X¹ and X² can be, independently, any moiety containing at least one carbon atom, wherein, when the substituted fullerene consists of a fullerene core and one or more (>CX¹X²) groups bonded thereto, at least one X¹ or X² moiety of at least one (>CX¹X²) group is not —COOH or the (>CX¹X²) groups are not in the C3 or D3 orientation on the fullerene core.

In one further embodiment, X¹ can be selected from —H, —COOH, —CONH₂, —CONHR′, —CONR′₂, —COOR′, —CHO, —(CH₂)_{d}OH, or a salt thereof, wherein each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol, and d is an integer from 0 to about 20. In another further embodiment, X¹ can be selected from —R, —RCOOH, —RCONH₂, —RCONHR′, —RCONR′₂, —RCOOR′, —RCHO, —R(CH₂)_{d}OH, or a salt thereof, wherein R is a hydrocarbon moiety having from 1 to about 6 carbon atoms.

Similarly, but independently, in one embodiment X² can be selected from —H, —COOH, —CONH₂, —CONHR′, —CONR′₂, —COOR′, —CHO, —(CH₂)_{d}OH, or a salt thereof. In another embodiment, X² can be selected from —R, —RCOOH, —RCONH₂, —RCONHR′, —RCONR′₂, —RCOOR′, —RCHO, —R(CH₂)_{d}OH, or a salt thereof.

All structural formulas shown herein are not to be construed as limiting the structure to any particular isomer.

All possible isomers of the substituted fullerenes disclosed herein are within the scope of the present disclosure. For example, in >CX¹X², one group (X¹ or X²) of each substituent points away from the fullerene core, and the other group points toward the fullerene core. Continuing the example, the central carbon of each substituent group (by which is meant the carbon with two bonds to the fullerene core, one bond to X¹, and one bond to X²) is chiral when X¹ and X² are different.

It will also be apparent that substituted fullerenes having two or more substituent groups will have isomers resulting from the different possible sites of bonding of the substituent groups to the fullerene core.

In one embodiment, the substituted fullerene is a decarboxylation product of (C₆₀(>C(COOH)₂)₃) (C3). By “decarboxylation product of C3” is meant the product of a reaction wherein 0 or 1 carboxy (—COOH) groups are removed from each of the three malonate moieties (>C(COOH)₂) of C3 and replaced with —H, provided at least one of the malonate moieties has 1 carboxy group replaced with —H. This can be considered as the loss of CO₂ from a malonate moiety. Decarboxylation can be performed by heating C3 in acid, among other techniques.

During decarboxylation of C3, only CO₂ loss from C3 is observed; each malonate moiety retains at least one carboxyls; and the decarboxylation stops at loss of 3 CO₂ groups, one from each malonate moiety of C3. The skilled artisan having the benefit of the present disclosure will recognize that substituted fullerenes having 1, 2, 4, 5, or 6 malonate moieties would also undergo decarboxylation.

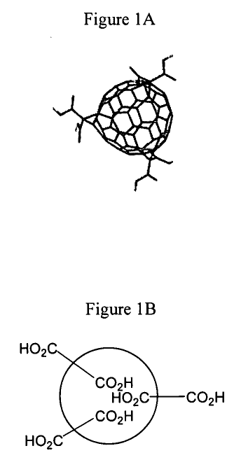

In C3, each malonate moiety has a carboxy group pointing to the outside (away from the fullerene), which we herein term exo, and a carboxy group pointing to the inside (toward the fullerene), which we herein term endo. FIG. 1A presents both a structural formula of C3 (FIG. 1A) and a diagram representing exo- and endo-acids (FIG. 1B).

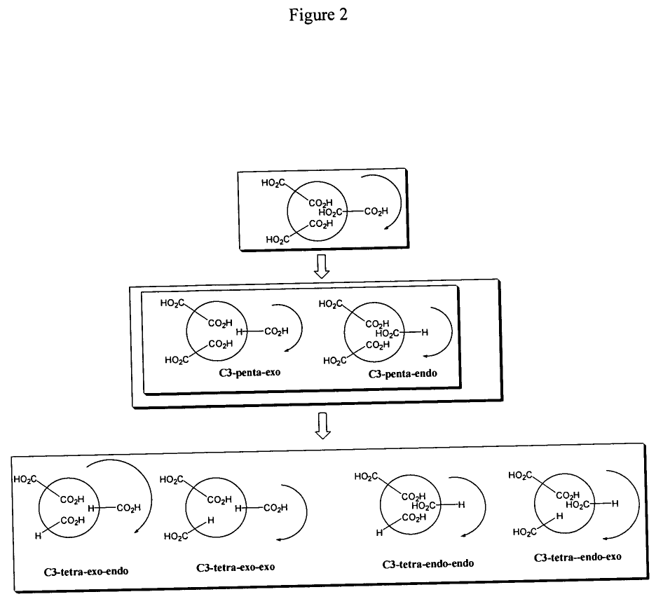

FIG. 2 shows C3 (in box 30) and the products of subsequent loss via decarboxylation of one or two CO₂ groups, giving C3-penta-acids (in box 32) and C3-tetra-acids (in box 34). Decarboxylation is represented by the open arrows 31 and 33; the isomers of C3, C3-penta-acid, and C3-tetra-acid are shown in box 30, in box 32, and in box 34, respectively.

In the interest of precise nomenclature, we define the order of exo or endo by always naming the groups in a clockwise manner.

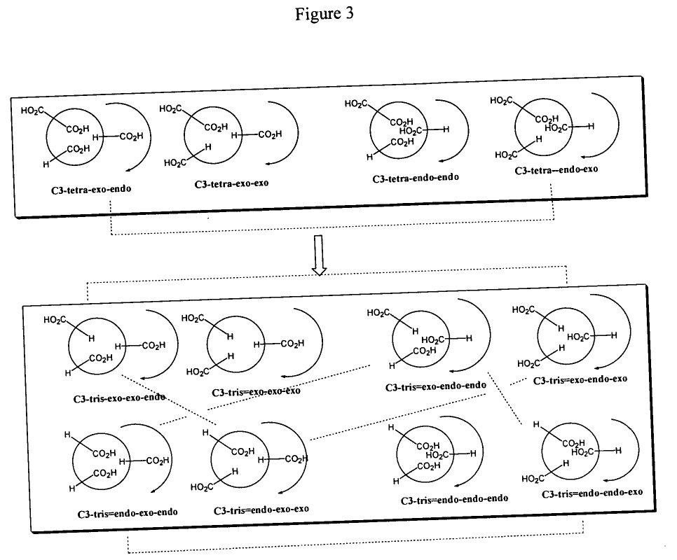

FIG. 3 shows the products of subsequent loss via decarboxylation of a third CO₂ group from the C3-tetra-acids shown in box 34, giving C3-tris-acids (box 42).

Decarboxylation is represented by the open arrow 41; the isomers of C3-tetra-acid and C3-penta-acid are shown in box 34 and in box 42, respectively. Isomers that differ only by rotation are connected by dashed lines 43, 44, and 45.

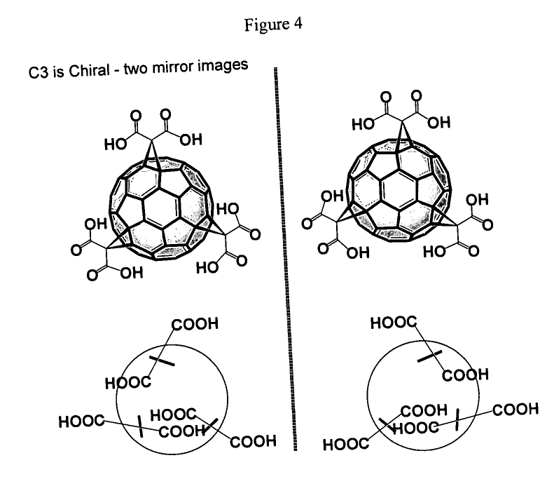

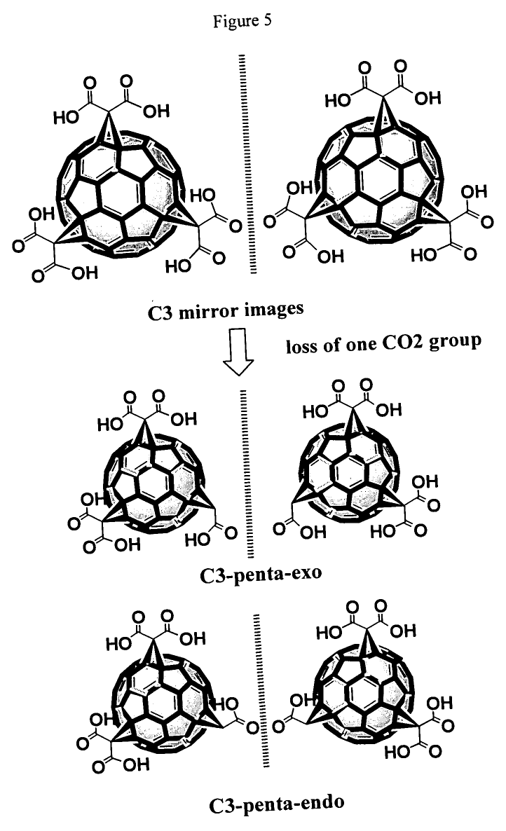

FIG. 4 shows the chirality of C3, both in a structural formula (mirror images 50a and 50b) and a schematic representation (mirror images 52a and 52b). FIG. 5 shows the chirality of C3-penta-acids (mirror images 60a and 60b; mirror images 62a and 62b).

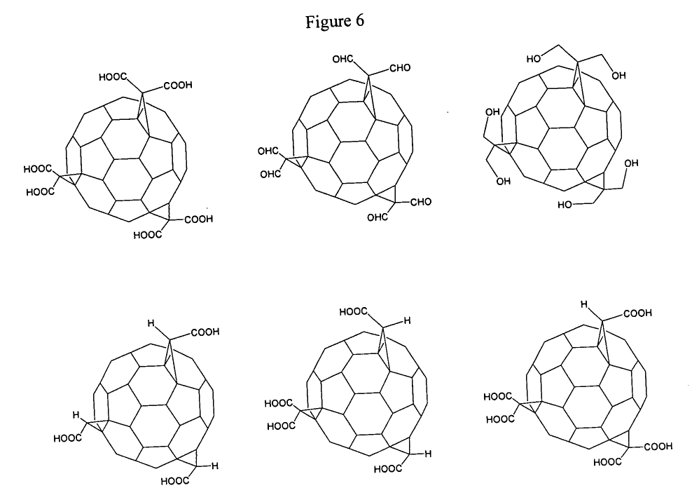

In another embodiment, the substituted fullerene comprises one of the structures 72, 74, 76, 77, or 78 shown in FIG. 6.

In one embodiment, the substituted fullerene comprises C₆₀ and 3 (>CX¹X²) groups in the C3 orientation or the D3 orientation. The D3 orientation is a mirror translation of the C3 orientation. The above description of C3-penta-acids, C3-tetra-acids, and C3-tris-acids also applies to D3 orientations of penta acids, tetra acids, and tris acids.

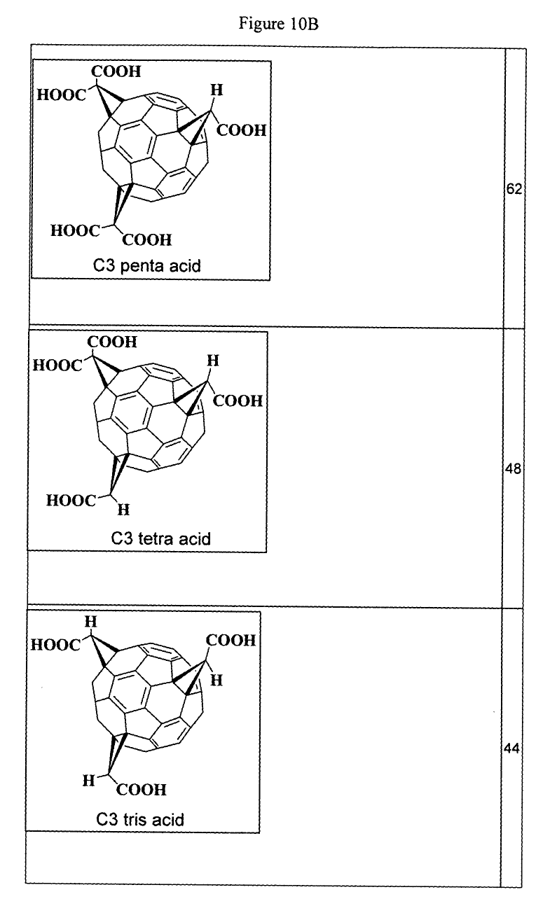

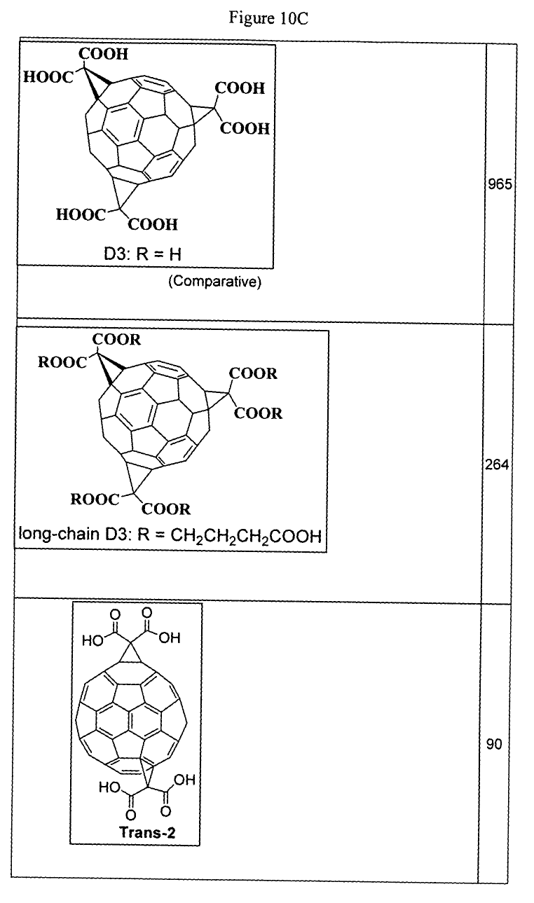

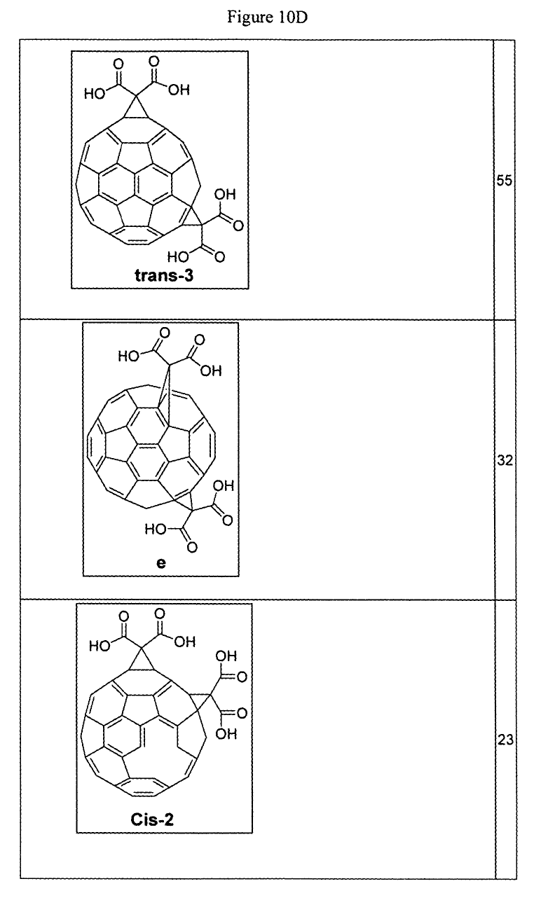

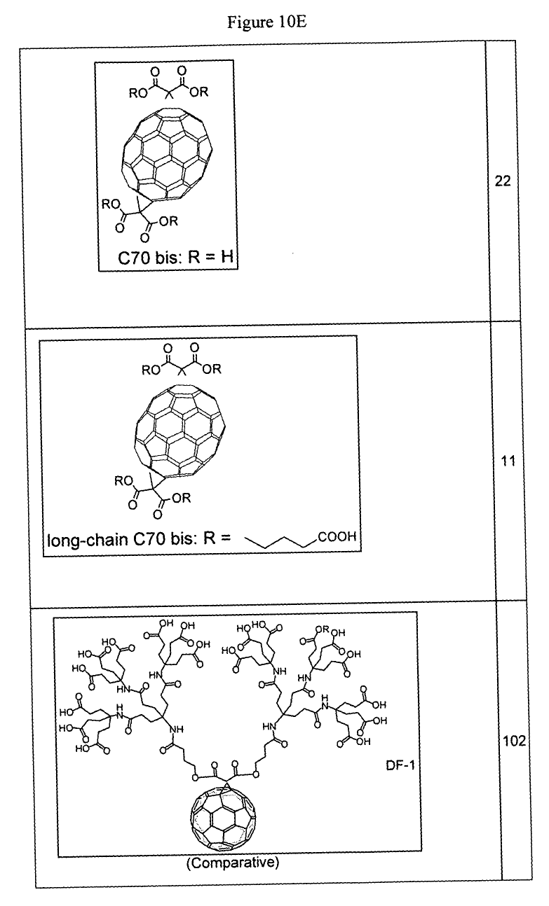

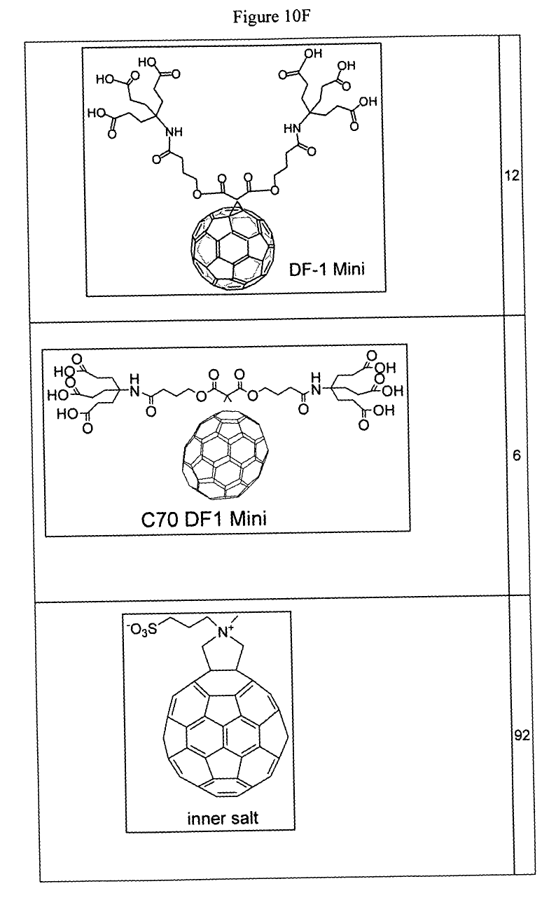

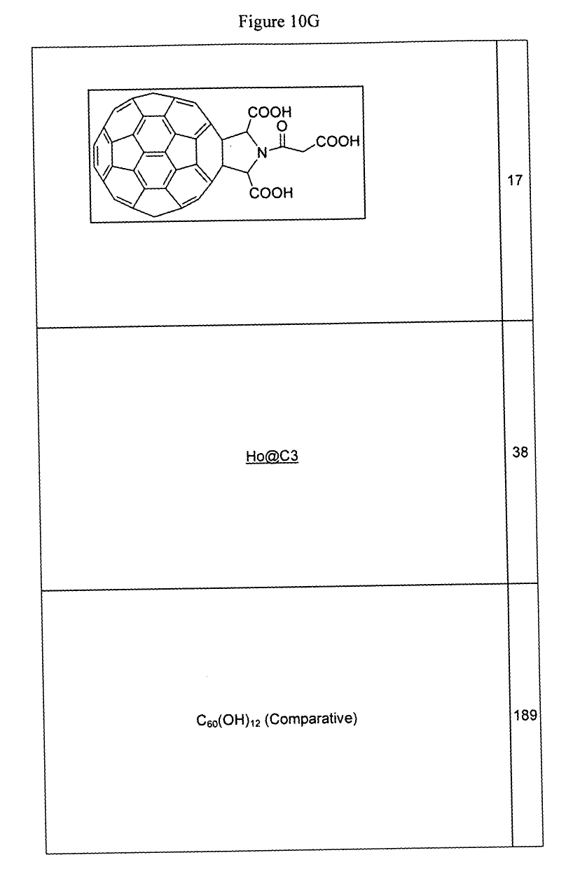

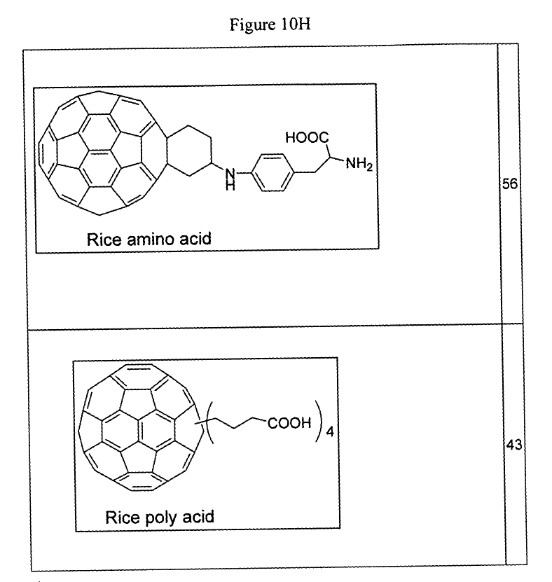

In one embodiment, as shown in FIG. 10, the substituted fullerene comprises C₆₀ and 2 (>CX¹X²) groups in the trans-2 orientation 1206, the trans-3 orientation 1207, the e orientation 1208, or the cis-2 orientation 1209.

In another embodiment, also as shown in FIG. 10, the substituted fullerene comprises C₇₀ and 2 (>CX¹X²) groups in the bis orientation 1210 or 1211.

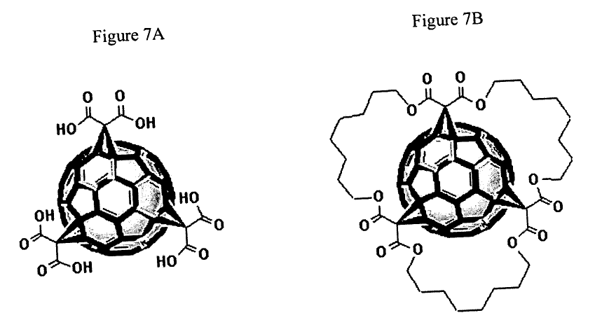

In one embodiment, the substituted fullerene has the formula C₆₀(>C(COOR)₂)ₙ, wherein —R is —H or an organic moiety comprising one or more carbon atoms, and n is an integer from 1 to 30. The —R moiety can be bonded to another —R moiety of the same or another substituent group. In another embodiment, the substituted fullerene has the structure shown in FIG. 7B.

In one embodiment, the substituted fullerene comprises a fullerene core (Cn) and from 1 to 18 —X³ groups bonded to the fullerene core. The notation “—X³” indicates the group is bonded to the fullerene core by a single bond between one atom of the X³ group and one carbon atom of the fullerene core. In specific X³ groups referred to below, any unfilled valences represent the single bond between the group and the fullerene core.

In one embodiment, the substituted fullerene comprises from 1 to about 6 —X³ groups and each —X³ group is independently selected from:

—N⁺(R²)(R³)(R⁴), wherein R², R³, and R⁴ are independently —H or —(CH₂)_{d}—CH₃, wherein d is an integer from 0 to about 20;

—N(R²)(R³)(R⁸), wherein R² and R³ are independently —H or —(CH₂)_{d}—CH₃, wherein d is an integer from 0 to about 20, and each R⁸ is independently —(CH₂)_{f}—SO₃⁻, —(CH₂)ᵣ—PO₄⁻, or —(CH₂)_{f}—COO⁻, wherein f is an integer from 1 to about 20;

—C(R⁵)(R⁶)(R⁷), wherein R⁵, R⁶, and R⁷ are independently —COOH, —H, —CH(═O), or —CH₂OH;

—C(R²)(R³)(R⁸), wherein R² and R³ are independently —H or —(CH₂)_{d}—CH₃, wherein d is an integer from 0 to about 20, and each R⁸ is independently —(CH₂)_{f}SO₃⁻, —(CH₂)_{f}—PO₄⁻, or —(CH₂)_{f}—COO⁻, wherein f is an integer from 1 to about 20;

—(CH₂)ₑ—COOH, —(CH₂)ₑ—CONH₂, —(CH₂)ₑ—COOR′, or a peptidyl moiety, wherein e is an integer from 1 to about 6 and each R¹ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol; or

an aromatic heterocyclic moiety containing a cationic nitrogen.

As will be apparent from the foregoing, a substituted fullerene according to this embodiment can comprise one or more groups selected from one or more of the foregoing categories.

In another embodiment, the substituted fullerene comprises a fullerene core (Cn) and from 1 to 6 —X⁴— groups bonded to the fullerene core. The notation “—X⁴—” indicates the group is bonded to the fullerene core by two single bonds, wherein one single bond is between a first atom of the group and a first carbon of the fullerene core, and the other single bond is between a second atom of the group and a second carbon of the fullerene core. (The adjectives “first” and “second,” wherever they appear herein, do not imply a particular ordering, in time, space, or both, of the nouns modified by the adjectives).

In one embodiment, each —X⁴— group is independently 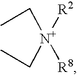
wherein R² is independently —H or —(CH₂)_{d}—CH₃, wherein d is an integer from 0 to about 20, and R⁸ is independently —(CH₂)_{f}—SO₃⁻, —(CH₂)_{f}—PO₄⁻, or —(CH₂)_{f}—COO⁻, wherein f is an integer from 1 to about 20.

In another embodiment, each —X⁴— group is independently 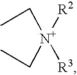
wherein each R² and R³ is independently —H or —(CH₂)_{d}—CH₃, wherein d is an integer from 0 to about 20.

In another embodiment, each —X⁴— group is independently selected from: 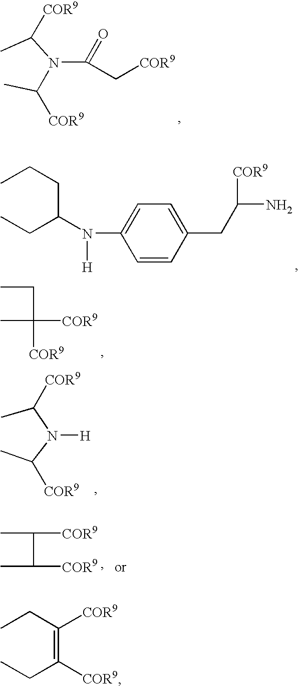
wherein each R² is independently —H or —(CH₂)_{d}—CH₃, wherein d is an integer from 0 to about 20, and each R⁹ is independently —H, —OH, —OR′, —NH₂, —NHR′, —NHR′₂, or —(CH₂)_{d}OH, wherein each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol.

In one embodiment of the present invention, the substituted fullerene comprises a fullerene core (Cn) and from 1 to 6 dendrons bonded to the fullerene core.

A dendron within the meaning of the invention is an addendum of the fullerene which has a branching at the end as a structural unit. Dendrons can be considered to be derived from a core, wherein the core contains two or more reactive sites. Each reactive site of the core can be considered to have been reacted with a molecule comprising an active site (in this context, a site that reacts with the reactive site of the core) and two or more reactive sites, to define a first generation dendron. First generation dendrons are within the scope of the term “dendron,” as used herein. Higher generation dendrons can be considered to have formed by each reactive site of the first generation dendron having been reacted with the same or another molecule comprising an active site and two or more reactive sites, to define a second generation dendron, with subsequent generations being considered to have been formed by similar additions to the latest generation. Although dendrons can be formed by the techniques described above, dendrons formed by other techniques are within the scope of “dendron” as used herein.

The core of the dendron is bonded to the fullerene by one or more bonds between (a) one or more carbons of the fullerene and (b) one or more atoms of the core. In one embodiment, the core of the dendron is bonded to the fullerene in such a manner as to form a cyclopropanyl ring.

In one embodiment, the core of the dendron comprises, between the sites of binding to the fullerene and the reactive sites of the core, a spacer, which can be a chain of 1 to about 100 atoms, such as about 2 to about 10 carbon atoms.

The generations of the dendron can comprise trivalent or polyvalent elements such as, for example, N, C, P, Si, or polyvalent molecular segments such as aryl or heteroaryl. The number of reactive sites for each generation can be about two or about three. The number of generations can be between 1 and about 10, inclusive.

More information regarding dendrons suitable for adding to fullerenes can be found in Hirsch, U.S. Pat. No. 6,506,928, the disclosure of which is hereby incorporated by reference.

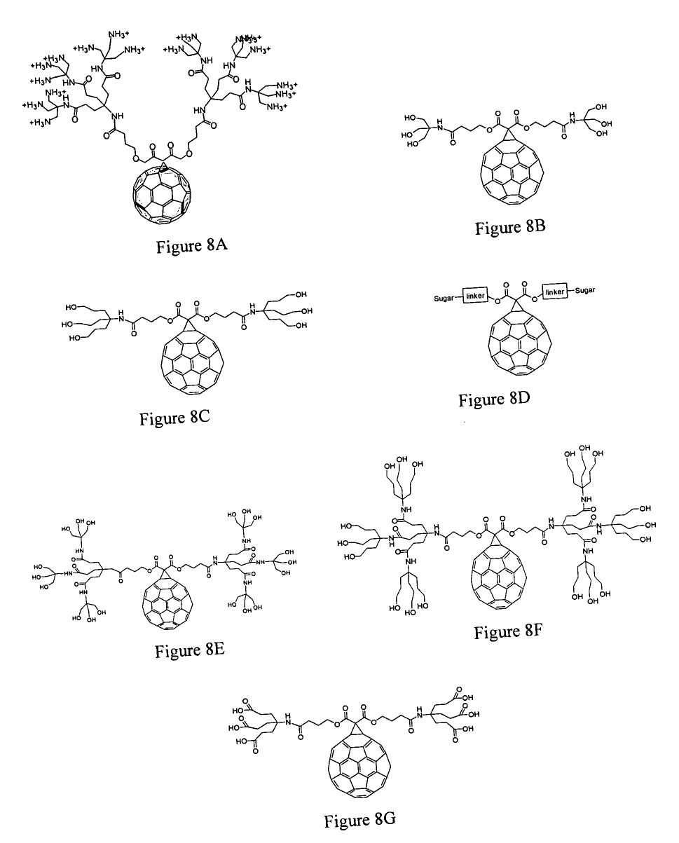

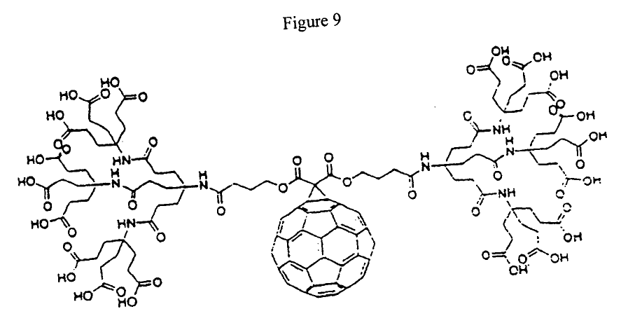

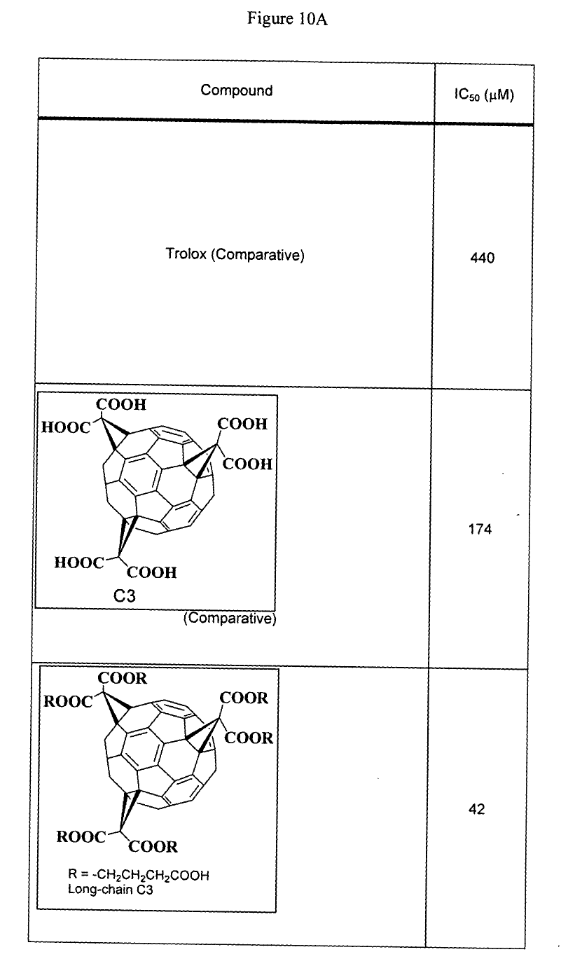

In a further embodiment, the substituted fullerene has a structure selected from FIGS. 8A-8G. In FIG. 8D, each “Sugar” independently represents a carbohydrate moiety, and each “linker” independently represents an organic or inorganic moiety. In a further embodiment, each Sugar is independently ribose or deoxyribose, and each “linker” independently has the formula —(CH₂)_{d}—, wherein d is an integer from 0 to about 20.

The substituted fullerene of this embodiment can further comprise a nondendron moiety, which is an addendum to a fullerene, wherein the addendum does not have a core and generations structure as found in dendrons defined above. Exemplary nondendrons include, but are not limited to, —COOH,—When the dendron comprises 18 —COOH groups, the substituted fullerene comprises one or more nondendrons.

The substituted fullerene of the present invention can satisfy one, two, or more of the foregoing embodiments, consistent with the plain meaning of “comprising.”

A substituted fullerene of any of the foregoing embodiments can further comprise an endohedral metal. “Metal” means at least one atom of a metallic element, and “endohedral” means the metal is encaged by the fullerene core. The metal can be elemental, or it can be an atom or atoms in a molecule comprising other elements. A substituted fullerene comprising an endohedral metal can be termed a “metallofullerene.” In a further embodiment, the metallofullerene can be represented by the structure:
Mₘ@Cₙ,
wherein each M independently is a molecule containing a metal;

m is an integer from 1 to about 5; and

Cₙ is a fullerene core comprising n carbon atoms, wherein n is an integer equal to or greater than 60.

In one embodiment, M is a transition metal atom. In one embodiment, M is a metal atom with an atomic number greater than about 55. Exemplary metals include those which do not form metal carbides. In one embodiment, the metal is Ho, Gd, or Lu.

In one embodiment, M is an organometallic molecule or an inorganometallic molecule. In one embodiment, M is a molecule having the formula M′₃N, wherein each M′ independently is a metal atom. Each metal atom M′ can be any metal, such as a transition metal, a metal with an atomic number greater than about 55, or one of the exemplary metals given above, among others.

In one embodiment, M is a metal capable of reacting with a reactive oxygen species.

In one embodiment, the metallofullerene is characterized in that M is Ho, Ho₃N, Gd, Gd₃N, Lu, or Lu₃N; m is 1; and n is 60.

In one embodiment, the substituted fullerene is polymerized, by which is meant a plurality of fullerene cores are present in a single molecule. The molecule can comprise carbon-carbon bonds between a first fullerene core and a second fullerene core, covalent bonds between a first substituent group on a first fullerene core and a second substituent group on a second fullerene core, or both.

The substituted fullerene can be used in the method as a composition with other components. In one embodiment, the composition further comprises an amphiphilic fullerene having the formula (B)_{b}-Cₙ-(A)ₐ, wherein Cₙ is a fullerene moiety comprising n carbon atoms, wherein n is an integer and 60≦n≦240; B is an organic moiety comprising from 1 to about 40 polar headgroup moieties; b is an integer and 1≦b≦5; each B is covalently bonded to the Cₙ through 1 or 2 carbon-carbon, carbon-oxygen, or carbon-nitrogen bonds; A is an organic moiety comprising a terminus proximal to the Cₙ and one or more termini distal to the Cₙ, wherein the termini distal to the Cₙ each comprise —CₓH_{y}, wherein x is an integer and 8≦x≦24, and y is an integer and 1≦y≦2x+1; a is an integer, 1≦a≦5; 2≦b+a≦6; and each A is covalently bonded to the Cₙ through 1 or 2 carbon-carbon, carbon-oxygen, or carbon-nitrogen bonds.

B is chosen from any organic moiety comprising from 1 to about 40 polar headgroup moieties. A “polar headgroup” is a moiety which is polar, by which is meant that the vector sum of the bond dipoles of each bond within the moiety is nonzero. A polar headgroup can be positively charged, negatively charged, or neutral. The polar headgroup can be located such that at least a portion of the moiety can be exposed to the environment of the molecule. Exemplary polar headgroup moieties can include, but are not limited to, carboxylic acid, alcohol, amide, and amine moieties, among others known in the art. Preferably, B has from about 6 to about 24 polar headgroup moieties. In one embodiment, B has a structure wherein the majority of the polar headgroup moieties are carboxylic acid moieties, which are exposed to water when the amphiphilic fullerene is dissolved in an aqueous solvent. A dendrimeric or other regular highly-branched structure is suitable for the structure of B.

The value of b can be any integer from 1 to 5. In one embodiment, if more than one B group is present (i.e., b>1), that all such B groups are adjacent to each other. By “adjacent” in this context is meant that no B group has only A groups, as defined below, and/or no substituent groups at all the nearest neighboring points of addition. In the case of an octahedral addition pattern when b>1, “adjacent” means that the four vertices of the octahedron in closest proximity to the B group are not all A groups or null.

In one embodiment, B comprises 18 polar headgroup moieties and b=1.

The polar headgroup moieties of B tend to make the B group or groups hydrophilic.

Each B is bonded to Cₙ through a covalent bond or bonds. Any covalent bond which a fullerene carbon is capable of forming and will not disrupt the fullerene structure is contemplated. Examples include carbon-carbon, carbon-oxygen, or carbon-nitrogen bonds. One or more atoms, such as one or two atoms, of the B group can participate in bonding to Cₙ. In one embodiment, one carbon atom of the B group is bonded to two carbon atoms of Cₙ, wherein the two carbon atoms of Cₙ are bonded to each other.

In one embodiment, B has the amide dendron structure
>C(C(═O)OC₃H₆C(═O)NHC(C₂H₄C(═O)NHC(C₂H₄C(═O)OH)₃)₃)₂.

In the amphiphilic fullerene, A is an organic moiety comprising a terminus proximal to the Cₙ and one or more termini distal to the Cₙ. In one embodiment, the organic moiety comprises two termini distal to Cₙ. By “terminus proximal to Cₙ” is meant a portion of the A group that comprises one or more atoms, such as one or two atoms, of the A group which form a bond or bonds to Cₙ. By “terminus distal to Cₙ” is meant a portion of the A group that does not comprise any atoms which form a bond or bonds to Cₙ, but that does comprise one or more atoms which form a bond or bonds to the terminus of the A group proximal to Cₙ.

Each terminus distal to the Cₙ comprises —CₓH_{y}, wherein x is an integer and 8≦x≦24, and y is an integer and 1≦y≦2x+1. The —CₓH_{y} can be linear, branched, cyclic, aromatic, or some combination thereof. Preferably, A comprises two termini distal to Cₙ, wherein each —CₓH_{y} is linear, 12≦x≦18, and y=2x+1. More preferably, in each of the two termini, x=12 and y=25.

The termini distal to Cₙ tend to make the A groups hydrophobic or lipophilic.

The value of a can be any integer from 1 to 5. Preferably, a is 5. In one embodiment, if more than one A group is present (i.e., a>1), all such A groups are adjacent to each other. By “adjacent” in this context is meant that no A group has only B groups, as defined below, and/or no substituent groups at all the nearest neighboring points of addition. In the case of an octahedral addition pattern, when a>1, “adjacent” means that the four vertices of the octahedron in closest proximity to the A group are not all B groups or null.

Each A is bonded to Cₙ through a covalent bond or bonds. Any covalent bond which a fullerene carbon is capable of forming and will not disrupt the fillerene structure is contemplated. Examples include carbon-carbon, carbon-oxygen, or carbon-nitrogen bonds. One or more atoms, such as one or two atoms, of the A group can participate in bonding to Cₙ. In one embodiment, one carbon atom of the A group is bonded to two carbon atoms of Cₙ, wherein the two carbon atoms of Cₙ are bonded to each other.

In one embodiment, A has the structure >C(C(═O)O(CH₂)₁₁CH₃)₂.

The number of B and A groups is chosen to be from 2 to 6, i.e., 2≦b+a≦6. In one embodiment, b+a=6. The combination of hydrophilic B group(s) and hydrophobic A group(s) renders the fullerene amphiphilic. The number and identity of B groups and A groups can be chosen to produce a fullerene with particular amphiphilic qualities which may be desirable for particular intended uses.

The amphiphilic fullerenes are capable of forming a vesicle, wherein the vesicle wall comprises the amphiphilic fullerene. A “vesicle,” as the term is used herein, is a collection of amphiphilic molecules, by which is meant, molecules which include both (a) hydrophilic (“water-loving”) regions, typically charged or polar moieties, such as moieties comprising polar headgroups, among others known to one of ordinary skill in the art, and (b) hydrophobic (“water-hating”) regions, typically apolar moieties, such as hydrocarbon chains, among others known to one of ordinary skill in the art. In aqueous solution, the vesicle is formed when the amphiphilic molecules form a wall, i.e., a closed three-dimensional surface. The wall defines an interior of the vesicle and an exterior of the vesicle. Typically, the exterior surface of the wall is formed by amphiphilic molecules oriented such that their hydrophilic regions are in contact with water, the solvent in the aqueous solution. The interior surface of the wall may be formed by amphiphilic molecules oriented such that their hydrophilic regions are in contact with water present in the interior of the vesicle, or the interior surface of the wall may be formed by amphiphilic molecules oriented such that their hydrophobic regions are in contact with hydrophobic materials present in the interior of the vesicle.

The amphiphilic molecules in the wall will tend to form layers, and therefore, the wall may comprise one or more layers of amphiphilic molecules. If the wall consists of one layer, it may be referred to as a “unilayer membrane” or “monolayer membrane.” If the wall consists of two layers, it may be referred to as a “bilayer membrane.” Walls with more than two layers, up to any number of layers, are also within the scope of the present invention.

The vesicle may be referred to herein as a “buckysome.”

In one embodiment, the vesicle wall is a bilayer membrane. The bilayer membrane comprises two layers, an interior layer formed from the amphiphilic fwllerene and other amphiphilic compound or compounds, if any, wherein substantially all the amphiphilic fullerene and other amphiphilic molecules are oriented with their hydrophobic portions toward the exterior layer, and an exterior layer formed from the amphiphilic fullerene and other amphiphilic compound or compounds, if any, wherein substantially all the amphiphilic fullerene and other amphiphilic molecules are oriented with their hydrophobic portions toward the interior layer. As a result, the hydrophilic portions of substantially all molecules of each of the interior and exterior layers are oriented towards aqueous solvent in the vesicle interior or exterior to the vesicle.

For further details on the amphiphilic fullerenes and vesicles made therefrom, see Hirsch et al., U.S. patent application Ser. No. 10/367,646, filed Feb. 14, 2003, for “Use of Buckysome or Carbon Nanotube for Drug Delivery,” which is incorporated herein by reference.

The method can be used to treat one or more hearing loss, collateral damage of chemotherapy, or mucositis.

We have discovered that substituted fullerenes have antioxidant properties and thus can inhibit ROS damage to cells exposed to ROS or cells wherein ROS are generated.

It is known that fullerenes are essentially non-toxic and non-allergenic, including in topical skin applications. For example, see Huczko et al., Fullerene Sci. Tech. 7(5):935-939 (1999).

In one embodiment, the substituted fullerene is a component of a composition also comprising a carrier. The carrier can be any material or plurality of materials which can form a composition with the substituted fullerene. The particular carrier can be selected by the skilled artisan in view of the intended use of the composition and the properties of the substituted fullerene, among other parameters apparent in light of the present disclosure.

The composition can comprise the substituted fullerene in any form. In one embodiment, the substituted fullerene is dispersed or dissolved in an aqueous solution suitable for subcutaneous administration or intravascular administration. In another embodiment, the substituted fullerene is dispersed or dissolved in a water-in-oil emulsion, a microemulsion, a multiple emulsion, a gel, or a fluorocarbon emulsion.

Non-limiting examples of particular carriers and particular compositions follow.

In one embodiment, the carrier is water, and the composition is an aqueous solution comprising water and the substituted fullerene. The composition can further comprise solutes, such as salts, acids, bases, or mixtures thereof, among others. The composition can also comprise a surfactant, an emulsifier, or another compound capable of improving the solubility of the substituted fullerene in water.

Other carriers will be apparent to the skilled artisan having the benefit of the present disclosure.

In one embodiment, the carrier is a pharmaceutically-acceptable carrier. By “pharmaceutically-acceptable” is meant that the carrier is suitable for use in medicaments intended for administration to a mammal. Parameters which may considered to determine the pharmaceutical acceptability of a carrier can include, but are not limited to, the toxicity of the carrier, the interaction between the substituted fullerene and the carrier, the approval by a regulatory body of the carrier for use in medicaments, or two or more of the foregoing, among others. In one embodiment, further components of the composition are pharmaceutically acceptable.

In addition to the substituted fullerene and the carrier, and further components described above, the composition can also further comprise other compounds, such as preservatives, adjuvants, excipients, binders, diluents, surfactants, or other adjuncts other agents capable of ameliorating one or more diseases, or mixtures thereof, among others. In one embodiment, the other compounds are pharmaceutically acceptable.

The concentration of the substituted fullerene in the composition can vary, depending on the carrier and other parameters apparent to the skilled artisan having the benefit of the present disclosure. A typical composition can comprise from about 0.01 wt % to about 5 wt % substituted fullerene. In one embodiment, the composition can comprise from about 0.1 wt % to about 0.5 wt % substituted fullerene. The concentration of other components of the composition can also vary along the same lines. Generally, the balance of the composition will comprise the carrier and other compounds.

The composition can be administered after diagnosis of a hearing loss, collateral damage of chemotherapy, or mucositis (i.e., the mammal is afflicted with the hearing loss, collateral damage of chemotherapy, or mucositis) or prophylactically to minimize the likelihood of acquiring a hearing loss, collateral damage of chemotherapy, or mucositis, the severity of the disease, or both (i.e., the mammal is potentially afflicted with the hearing loss, collateral damage of chemotherapy, or mucositis). An example of a mammal potentially afflicted with collateral damage of chemotherapy is an H. sapiens intending to be exposed to cytotoxic drugs in the course of a cancer treatment.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Superoxide radicals were generated by employing the xanthine/xanthine oxidase/cytochrome c system. The reaction was initiated by the addition of xanthine oxidase (7.5×10⁻³ units) to the incubation mixture and the reaction was followed in terms of the reduction of cytochrome c and the corresponding increase in the absorbance at 550 nm. The reduction of ferricytochrome c into ferrocytochrome c was determined using the molar absorption coefficients of 9 mmol⁻¹ cm⁻¹ and 27.7 mmol⁻¹ cm⁻¹ for the oxidized and reduced forms, respectively. All assays were performed at room temperature. The incubation mixture consisted of 50 mM potassium phosphate, 0.1 mM EDTA, 0.01 mM cytochrome c, and 0.05 mM xanthine, along with the indicated concentration of antioxidant. A total volume of 3 mL was used in each experiment.

Various substituted fullerenes, both those known in the art and those reported herein, were tested, as shown in FIG. 10. Trolox, a known non-fullerene antioxidant, was tested as a comparative example. A negative control (without antioxidant, not shown) was run to establish a baseline for the reduction of cytochrome c. The compounds and their IC₅₀ values are given in FIG. 10. Comparative compounds are indicated with the notation “(Comparative).”

Of the comparative compounds, DF-1 had the lowest IC₅₀, 102 μm. However, many of the compounds of the present invention had much lower IC₅₀ values, indicating higher antioxidant properties.

All of the compositions and the methods disclosed and claimed herein can be made and executed without undue experimentation in light of the present disclosure.

While the compositions and methods of this invention have been described in terms of particular embodiments, it will be apparent to those of skill in the art that variations may be applied to the compositions and the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of ameliorating a hearing loss, collateral damage of chemotherapy, or mucositis in a mammal, comprising:
administering a composition comprising a substituted fullerene to the mammal afflicted with the hearing loss, collateral damage of chemotherapy, or mucositis or potentially afflicted with the hearing loss, collateral damage of chemotherapy, or mucositis, wherein the substituted fullerene comprises a fullerene core (Cn) and at least one of:
(i) from 1 to 3 (>CX¹X²) groups bonded to the fullerene core;
(ii) from 1 to 18 —X³ groups bonded to the fullerene core;
(iii) from 1 to 6 —X⁴ groups bonded to the fullerene core; or
(iv) from 1 to 6 dendrons bonded to the fullerene core.

2. The method of claim 1, wherein the substituted fullerene comprises a fullerene core (Cn) having 60 carbon atoms or 70 carbon atoms.

3. The method of claim 1, wherein each X¹ and X² is independently selected from —H, —COOH, —CONH₂, —CONHR′, —CONR′₂, —COOR′, —CHO, —(CH₂)_{d}OH, —R, —RCOOH, —RCONH₂, —RCONHR′, —RCONR′₂, —RCOOR′, —RCHO, —R(CH₂)_{d}OH, or a salt thereof, wherein each R is a hydrocarbon moiety having from 1 to about 6 carbon atoms and each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol, and d is an integer from 0 to about 20.

4. The method of claim 1, wherein the substituted fullerene comprises C₆₀ and 3 (>CX¹X²) groups in the C3 orientation or the D3 orientation.

5. The method of claim 1, wherein the substituted fullerene comprises C₆₀ and 2 (>CX¹X²) groups in the trans-2 orientation, the trans-3 orientation, the e orientation, or the cis-2 orientation.

6. The method of claim 1, wherein the substituted fullerene comprises C₇₀ and 2 (>CX¹X²) groups in the bis orientation.

7. The method of claim 1, wherein the substituted fullerene has the structure shown in FIG. 7B.

8. The method of claim 1, wherein the substituted fullerene comprises from I to about 6 —X³ groups and each —X³ group is independently selected from:
—N⁺(R²)(R³)(R⁴), wherein R², R³, and R⁴ are independently —H or —(CH₂)_{d}—CH₃, wherein d is an integer from 0 to about 20;
—N(R²)(R³)(R⁸), wherein R² and R³ are independently —H or —(CH₂)_{d}—CH₃, wherein d is an integer from 0 to about 20, and each R⁸ is independently —(CH₂)_{f}—SO₃⁻, —(CH₂)_{f}—PO₄⁻, or —(CH₂)_{f}—COO⁻, wherein f is an integer from 1 to about 20;
—C(R⁵)(R⁶)(R⁷), wherein R⁵, R⁶, and R⁷ are independently —COOH, —H, —CH(═O), or —CH₂OH;
—C(R²)(R³)(R⁸), wherein R² and R³ are independently —H or —(CH₂)_{d}—CH₃, wherein d is an integer from 0 to about 20, and each R⁸ is independently —(CH₂)_{f}—SO₃⁻, —(CH₂)_{f}—PO₄⁻, or —(CH₂)_{f}—COO²⁰, wherein f is an integer from 1 to about 20;
—(CH₂)ₑ—COOH, —(CH₂)ₑ—CONH₂, —(CH₂)ₑ—COOR′, or a peptidyl moiety, wherein e is an integer from 1 to about 6 and each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol; or
an aromatic heterocyclic moiety containing a cationic nitrogen.

9. The method of claim 1, wherein each —X⁴— group is independently 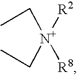
wherein R² is independently —H or —(CH₂)_{d}—CH₃, wherein d is an integer from 0 to about 20, and R⁸ is independently —(CH₂)_{f}—SO₃⁻, —(CH₂)_{f}—PO₄⁻, or —(CH₂)_{f}—COO⁻, wherein f is an integer from 1 to about 20.

10. The method of claim 1, wherein each —X⁴— group is independently 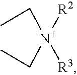
wherein each R² and R³ is independently —H or —(CH₂)_{d}—CH₃, wherein d is an integer from 0 to about 20.

11. The method of claim 1, wherein each —X⁴— group is independently 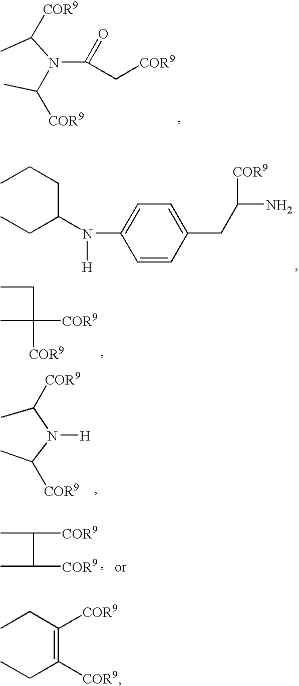
wherein each R² is independently —H or —(CH₂)_{d}—CH₃, wherein d is an integer from 0 to about 20, and each R⁹ is independently —H, —OH, —OR′, —NH₂, —NHR′, —NHR′₂, or —(CH₂)_{d}OH, wherein each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol.

12. The method of claim 1, wherein the substituted fullerene has a structure selected from FIGS. 8A-8G.

13. The method of claim 1, wherein the substituted fullerene comprises an endohedral metal.

14. The method of claim 1, wherein the composition further comprises an amphiphilic fullerene having the formula (B)_{b}-Cₙ-(A)ₐ, wherein Cₙ is a fullerene moiety comprising n carbon atoms, wherein n is an integer and 60≦n≦240; B is an organic moiety comprising from 1 to about 40 polar headgroup moieties; b is an integer and 1≦b≦5; each B is covalently bonded to the Cₙ through 1 or 2 carbon-carbon, carbon-oxygen, or carbon-nitrogen bonds; A is an organic moiety comprising a terminus proximal to the Cₙ and one or more termini distal to the Cₙ, wherein the termini distal to the Cₙ each comprise —CₓH_{y}, wherein x is an integer and 8≦x≦24, and y is an integer and 1≦y≦2x+1; a is an integer, 1≦a≦5; 2≦b+a≦6; and each A is covalently bonded to the Cₙ through 1 or 2 carbon-carbon, carbon-oxygen, or carbon-nitrogen bonds.

15. A composition for ameliorating a hearing loss, collateral damage of chemotherapy, or mucositis in a mammal, comprising:
a substituted fullerene; and
a carrier
wherein the substituted fullerene comprises a fullerene core (Cn) and at least one of:
(i) from 1 to 3 (>CX¹X²) groups bonded to the fullerene core;
(ii) from 1 to 18 —X³ groups bonded to the fullerene core;
(iii) from 1 to 6 —X⁴— groups bonded to the fullerene core; or
(iv) from 1 to 6 dendrons bonded to the fullerene core.

16. The composition of claim 15, wherein each X¹ and X² is independently selected from —H, —COOH, —CONH₂, —CONHR′, —CONR′₂, —COOR′, —CHO, —(CH₂)_{d}OH, —R, —RCOOH, —RCONH₂, —RCONHR′, —RCONR′₂, —RCOOR′, —RCHO, —R(CH₂)_{d}OH, or a salt thereof, wherein each R is a hydrocarbon moiety having from 1 to about 6 carbon atoms and each R′ is independently (i) a hydrocarbon moiety having from 1 to about 6 carbon atoms, (ii) an aryl-containing moiety having from 6 to about 18 carbon atoms, (iii) hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol, and d is an integer from 0 to about 20.

17. The composition of claim 15, wherein the substituted fullerene has the structure shown in FIG. 7B.

18. The composition of claim 15, wherein the substituted fullerene comprises from 1 to about 6 —X³ groups and each —X³ group is independently selected from:
—N⁺(R²)(R³)(R⁴), wherein R², R³, and R⁴ are independently —H or —(CH₂)_{d}—CH₃, wherein d is an integer from 0 to about 20;
—N(R²)(R³)(R⁸), wherein R² and R³ are independently —H or —(CH₂)_{d}—CH₃, wherein d is an integer from 0 to about 20, and each R⁸ is independently —(CH₂)_{f}—SO₃⁻, —(CH₂)_{f}—PO₄⁻, or —(CH₂)_{f}—COO⁻, wherein f is an integer from 1 to about 20;
—C(R⁵)(R⁶)(R⁷), wherein R⁵, R⁶, and R⁷ are independently —COOH, —H, —CH(═O), or —CH₂OH;
—C(R²)(R³)(R⁸), wherein R² and R³ are independently —H or —(CH₂)_{d}—CH₃, wherein d is an integer from 0 to about 20, and each R⁸ is independently —(CH₂)_{f}—SO₃⁻, —(CH₂)_{f}—PO₄⁻, or —(CH₂)_{f}—COO⁻, wherein f is an integer from 1 to about 20;
—(CH₂)ₑ—COOH, —(CH₂)ₑ—CONH₂, —(CH₂)ₑ—COOR′, or a peptidyl moiety, wherein e is an integer from 1 to about 6 and each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol; or
an aromatic heterocyclic moiety containing a cationic nitrogen.

19. The composition of claim 15, wherein each —X⁴— group is independently 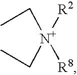
wherein R² is independently —H or —(CH₂)_{d}—CH₃, wherein d is an integer from 0 to about 20, and R⁸ is independently —(CH₂)_{f}—SO₃⁻, —(CH₂)_{f}—PO₄⁻, or —(CH₂)_{f}—COO⁻, wherein f is an integer from 1 to about 20.

20. The composition of claim 15, wherein each —X⁴— group is independently 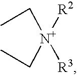
wherein each R² and R³ is independently —H or —(CH₂)_{d}—CH₃, wherein d is an integer from 0 to about 20.

21. The composition of claim 15, wherein each —X⁴— group is independently 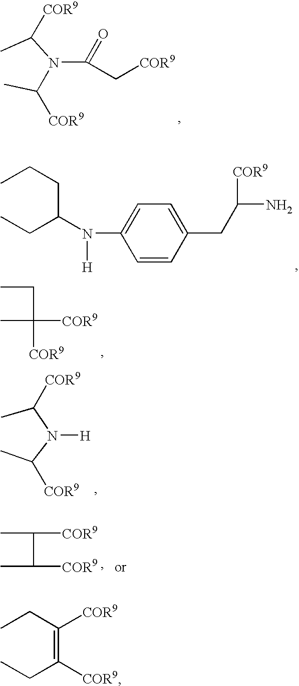
wherein each R² is independently —H or —(CH₂)_{d}—CH₃, wherein d is an integer from 0 to about 20, and each R⁹ is independently —H, —OH, —OR′, —NH₂, —NHR′, —NHR′₂, or —(CH₂)_{d}OH, wherein each R′ is independently (i) a hydrocarbon moiety having from 1 to about 6 carbon atoms, (ii) an aryl-containing moiety having from 6 to about 18 carbon atoms, (iii) a hydrocarbon moiety having from 1 to about 6 carbon atoms and a terminal carboxylic acid or alcohol, or (iv) an aryl-containing moiety having from 6 to about 18 carbon atoms and a terminal carboxylic acid or alcohol.

22. The composition of claim 15, wherein the substituted fullerene has a structure selected from FIGS. 8A-8G.
